(19) United States
(12) Patent Application Publication
(10) Pub. No.: US 2019/0070267 A1
(43) Pub. Date: Mar. 7, 2019

(54) DEVICE FOR DRUG RECONSTITUTION AND DELIVERY

(71) Applicant: Eighty Eight Pharma, Inc., Foxboro, MA (US)

(72) Inventor: Matthew Iorio, Foxboro, MA (US)

(21) Appl. No.: 15/694,244

(22) Filed: Sep. 1, 2017

(51) Int. Cl. A61K 38/40; A61K 9/08; A61J 1/20; A61J 1/18; A61J 1/16

(52) U.S. Cl. CPC A61K 38/40; A61K 9/08; A61J 1/201; A61J 1/10; A61J 1/2048; A61J 1/16; A61J 2200/76; A61J 1/18

(57) ABSTRACT

A device houses a lyophilized drug that is to be reconstituted and administered to a patient that has a disease. The device has a transparent cylinder that has a volume of at least 250 mL and that is configured to house the lyophilized drug. The cylinder has a first end that has a first spike, and the first end is opposite a second end that has a port. The first spike is configured to pierce an IV fluid bag and to form a fluid connection between the cylinder and the IV fluid bag. The port is configured to be pierced by a second spike that is part of a tubing, thereby forming a fluid connection between the cylinder and the tubing. At least a portion of the volume of the cylinder is occupied by at least 5 grams of the lyophilized drug.

### FIELD OF THE INVENTION

The invention generally relates to devices and methods for delivering drugs to a patient and, more particularly, the invention relates to delivering large dosages of apotransferrin intravenously.

### BACKGROUND OF THE INVENTION

Atransferrinemia is an autosomal recessive metabolic disorder in which there is an absence of transferrin in the blood. Transferrin is a plasma protein that transports iron through the blood. Atransferrinemia can lead to serious conditions such as heart failure or death.

### SUMMARY OF VARIOUS EMBODIMENTS

In accordance with one embodiment of the invention, a device houses a lyophilized drug that is to be reconstituted and administered to a patient that has a disease. The device has a transparent cylinder that has a volume of at least 250 mL and that is configured to house the lyophilized drug. The cylinder has a first end that has a first spike, and the first end is opposite a second end that has a port. The first spike is configured to pierce an IV fluid bag and to form a fluid connection between the cylinder and the IV fluid bag. The port is configured to be pierced by a second spike that is part of a tubing, thereby forming a fluid connection between the cylinder and the tubing. At least a portion of the volume of the cylinder is occupied by at least 5 grams of the lyophilized drug.

In some embodiments, the drug may be apotransferrin, transferrin, or a recombinant protein replacement for apotransferrin and may be provided as an apotransferrin preparation. The apotransferrin preparation may be recombinant apotransferrin. Preferably, apotransferrin may be at a mass of between about 5 grams and 20 grams. The disease may be atransferrinemia, hepatic failure, thalassemia, iron overload, hemochromatosis, or age-related macular degeneration. Furthermore, atransferrinemia may be congenital atransferrinemia.

Preferably, the device may be provided in a sterile packaging and may have a premeasured amount of the drug in the cylinder. Moreover, the device may be provided in tamper-evident packaging.

Furthermore, the cylinder may be fluidly connected with the IV fluid bag. This connection facilitates the reconstitution of the lyophilized drug. The tubing may be connected to a patient. To facilitate delivery of the drug to the patient, the cylinder may be fluidly connected with the tubing connected to the patient.

Among other things, the cylinder of the device may be formed from polypropylene. The cylinder may be manufactured in one or more pieces and assembled before use. The cylinder of the device may also have volume graduation markings. In some embodiments, the volume of the cylinder may be between about 250 mL and about 1000 mL, and the cylinder may have graduation markings in 50 mL increments from 0-1000 mL.

In addition, the device may have a fluid control mechanism to control the rate of fluid flow into the device. To support the weight of the device when it is filled with a solution (e.g., from the IV bag), the device may also have a hanging mechanism.

In accordance with another embodiment, a method of reconstituting a lyophilized drug that is administered to a patient that has a disease provides a device that has a transparent cylinder. The transparent cylinder has a volume of at least 250 mL and houses at least 5 grams of the lyophilized drug. The cylinder has a first end that has a first spike, and the first end is opposite a second end that has a port. The first spike is configured to pierce an IV fluid bag and to form a fluid connection between the cylinder and the IV fluid bag. The port is configured to be pierced by a second spike that is part of a tubing, thereby forming a fluid connection between the cylinder and the tubing. The first spike pierces the standard IV fluid bag and forms a fluid connection between the cylinder and the IV fluid bag, reconstituting the drug in the cylinder to form a reconstituted drug.

In another embodiment, a method of administering a drug to a patient uses the device described above. The method includes connecting the tubing to a patient, and piercing the port of the device using the second spike, which is part of the tubing. Thus, the cylinder and the tubing are fluidly connected and able to deliver the reconstituted drug to the patient.

In accordance with another embodiment a method of treating a patient that exhibits a disease includes administering an effective amount of an apotransferrin preparation to the patient in need of treatment using the device described above. The preparation has about 1-20 grams of apotransferrin.

Preferably, treatment may produce a serum transferrin level in the patient of about 150-400 mg/dL. In addition, treatment may produce a serum hemoglobin level in the patient of at least 10 g/dL. The treatment may be administered, for example, to the patient once every week or once every two weeks and the duration of treatment may be for life.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows one use of a drug delivery device configured in accordance with illustrative embodiments of the present invention.

FIG. 2 schematically shows the drug delivery device of FIG. 1.

FIG. 3 shows a process of delivering a drug to a patient in accordance with illustrative embodiments of the invention

### DESCRIPTION OF ILLUSTRATIVE EMBODIMENTS

In illustrative embodiments, a drug delivery device is placed in-line with an IV fluid bag to facilitate delivery of large drug dosages to a patient. The device meets the needs of the art by providing large dosages of transferrin that could not previously be delivered to a patient via standard methods, such as whole blood plasma transfusions. The advantages further include delivering high-dosages of transferrin efficiently, as compared to multiple injections, and the device introduces high levels of transferrin without causing an iron overload in the patient. The method of the invention also provides patients with a less invasive and more convenient mode of treatment by avoiding repeated injections and therefore repeated visits to the medical professional.

To that end, the device has a spike that is configured to pierce the port of an IV bag. The device also has a port that is configured to be pierced by a spike of an IV tubing. Thus, the device may be integrated in-line with a standard IV connection (e.g., using a standard IV fluid bag and standard tubing that connects to the IV fluid bag). The device has a large-volume container that may come preloaded with a drug, for example, apotransferrin, transferrin, or a recombinant derived transferrin replacement. The fluid from the IV fluid bag enters the large-volume container and reconstitutes the drug in the device. The reconstituted drug may then be delivered to the patient. Details of illustrative embodiments are discussed below.

Illustrative embodiments of the invention may be useful in the treatment of diseases such as atransferrinemia. Atransferrinemia is a rare, autosomal recessive, genetic disorder in which the individual fails to make transferrin, an iron-binding plasma protein that is involved in regulating the level of free iron in the blood and bodily fluids (see, generally, Hamill et al. Am J Clin Pathol. 1991 August; 96(2):215-8 and Bartnikas Biometals. 2012 Aug.; 25(4):677-86). While healthy adults typically have serum transferrin levels of about 170-370 mg/dL, patients suffering from atransferrinemia have lower than normal serum transferrin levels.

The liver is the primary site of transferrin synthesis, although transferrin is also produced in other organs and tissues throughout the body, such as the brain. Symptoms of atransferrinemia include anemia, hepatic abnormalities, arthritis, and recurrent infections and growth delays. Atransferrinemia and a milder form of atransferrinemia, hypo transferrinemia, are caused by mutations of the transferrin (TF) gene.

Individuals affected by atransferrinemia typically develop microcytic hypochromic anemia, which is characterized by abnormally small red blood cells containing insufficient levels of hemoglobin. Patients experiencing microcytic hypochromic anemia often present with fatigue and some individuals may present with an enlarged liver (hepatomegaly).

Because atransferrinemia causes iron accumulation in the body, the symptoms of atransferrinemia depend upon the location and extent of this iron accumulation. For example, atransferrinemia can affect the liver, heart, joints, pancreas, kidneys, and thyroid. The accumulation of iron in these organs due to atransferrinemia can result in damage such as cirrhosis of the liver, arthritis, hypothyroidism, and heart abnormalities. Severe cases of atransferrinemia can result in life-threatening complications such as pneumonia or an impaired ability to circulate blood to the lungs and the rest of the body, which results in congestive heart failure due to fluid buildup throughout the body.

A diagnosis of atransferrinemia may be made upon identification of the characteristic symptoms of atransferrinemia discussed above, the patient's family history, through genetic testing, and by determining whether the patient's serum transferrin levels are below the normal range of 170-370 mg/dL.

Patients suffering from atransferrinemia may benefit from treatment with apotransferrin (non-iron-bound transferrin), transferrin, recombinantly derived protein replacements for transferrin, and the transfusion of whole blood plasma. Because the liver is the primary site of transferrin synthesis, patients suffering from hepatic failure may also benefit from treatment with apotransferrin or transferrin. Furthermore, apotransferrin or transferrin may be useful in treating patients suffering from thalassemia and age-related macular degeneration (Brandsma M E et al. Biotechnol Adv. 2011; 29:230-238).

Apotransferrin and transferrin may be obtained and purified from several sources. In some embodiments, apotransferrin or transferrin is obtained and purified from human whole blood serum using methods well-known in the art (see, e.g., Bates and Schlabach J Biol Chem. 1973 May 10; 248(9):3228-32 and Aisen J Biol Chem. 1966 Apr. 25; 241(8):1666-71). In other embodiments, a protein replacement for apotransferrin or transferrin is produced and purified from a recombinant source by expressing apotransferrin or transferrin from a nucleic acid expression vector (see, e.g., U.S. Pat. No. 5,026,651 and Finnis Microb Cell Fact. 2010 Nov. 17; 9:87). For example, Optiferrin® (InVitria) is a commercially available recombinant transferrin produced from rice. Non-limiting examples of suitable recombinant sources include plants (e.g., rice, wheat, soybeans), mammalian cell cultures (e.g., CHO cells, COS-1 cells, HEK293 cells, and other mammalian cell cultures well-known in the art), bacteria (e.g., Escherichia coli and other bacteria well-known in the art), or yeast (e.g., Saccharomyces cerevisiae, Schizosaccharomyces pombe, and other yeast well-known in the art). Purified apotransferrin, transferrin, or recombinantly derived replacements for transferrin may be lyophilized or dried by other methods, such as spray drying, or air drying, and reconstituted with a fluid before use. Transferrin is currently used as a reagent in cell cultures. However, there are no approved medical uses for transferrin. In some embodiments, transferrin, apotransferrin, or a recombinantly derived protein replacement for transferrin for cell culture use is purified and processed according to current good manufacturing processes in order to be suitable for use in a human patient.

As used herein, “a disease” refers to a condition in which a patient presents serum transferrin levels below about 170 mg/dL, a condition in which an increase in serum transferrin levels ameliorates symptoms of the disease, or a condition in which a patient presents serum hemoglobin levels below 12 mg/dL caused by low transferrin levels. Non-limiting examples of such diseases include a transferrinemia, thalassemia, hepatic failure, iron overload, hemochromatosis, and age-related macular degeneration.

As used herein, “treating a disease,” means alleviating or ameliorating one or more symptoms of the disease.

“Treatment”, or “treating”, is defined as the application or administration of a preparation of apotransferrin, transferrin, or a recombinantly derived protein replacement for transferrin to a subject or patient who has a disease, with the purpose to cure, heal, alleviate, relieve, alter, remedy, ameliorate, improve or affect the symptoms of the disease by increasing serum transferrin levels and/or serum hemoglobin levels. The term “treatment” or “treating” is also used herein in the context of administering a preparation of apotransferrin, transferrin, or a recombinantly derived protein replacement for transferrin prophylactically.

The term “effective amount” is defined as an amount of apotransferrin, transferrin, or a recombinantly derived protein replacement for transferrin sufficient to achieve increased serum transferrin levels and/or serum hemoglobin levels, resulting in a complete or at least partial resolution of the symptoms of the disease.

As used herein, “increase” or “increasing” means 1.25, 1.5, 1.75, 2, 3, 4, 5, 10, 15, 20, 25, 30, 35, 40, 45, 50, 100, 500, 1000 or 10,000-fold more after administration of a preparation of apotransferrin, transferrin, or a recombinantly derived protein replacement for transferrin as compared to before administration of a preparation of apotransferrin, transferrin, or a recombinantly derived protein replacement for transferrin.

As used herein, “increase” or “increasing” also means 1, 5, 10, 15, 20, 25, 30, 35, 40, 45, 50, 55, 60, 65, 70, 75, 80, 85, 90, 95, 99 or 100% more after administration of a preparation of apotransferrin, transferrin, or a recombinantly derived protein replacement for transferrin as compared to before administration of a preparation of apotransferrin, transferrin, or a recombinantly derived protein replacement for transferrin.

As used herein “monitor,” “monitored,” or “monitoring” mean determining whether, following treatment of a subject diagnosed with a disease, the subject has been treated such that the subject's serum transferrin and/or hemoglobin levels have increased.

As used herein, a “recombinant protein replacement for apotransferrin” or a “recombinant protein replacement for transferrin” means a recombinantly produced protein capable of transporting iron in biological fluids and which may serve as a substitute for naturally produced transferrin in vivo. Non-limiting examples include recombinantly produced transferrin and fragments thereof, as well as recombinantly produced apotransferrin and fragments thereof, such as, Optiferrin® (InVitria).

Patients suffering from a transferrinemia, hepatic failure, thalassemia, iron overload, hemochromatosis, or age-related macular degeneration may require treatment with large doses of apotransferrin or transferrin, often for life. Illustrative embodiments of the drug delivery device may be used to deliver large doses of apotransferrin, transferrin, a recombinantly derived protein replacement for transferrin, and/or other drugs requiring reconstitution.

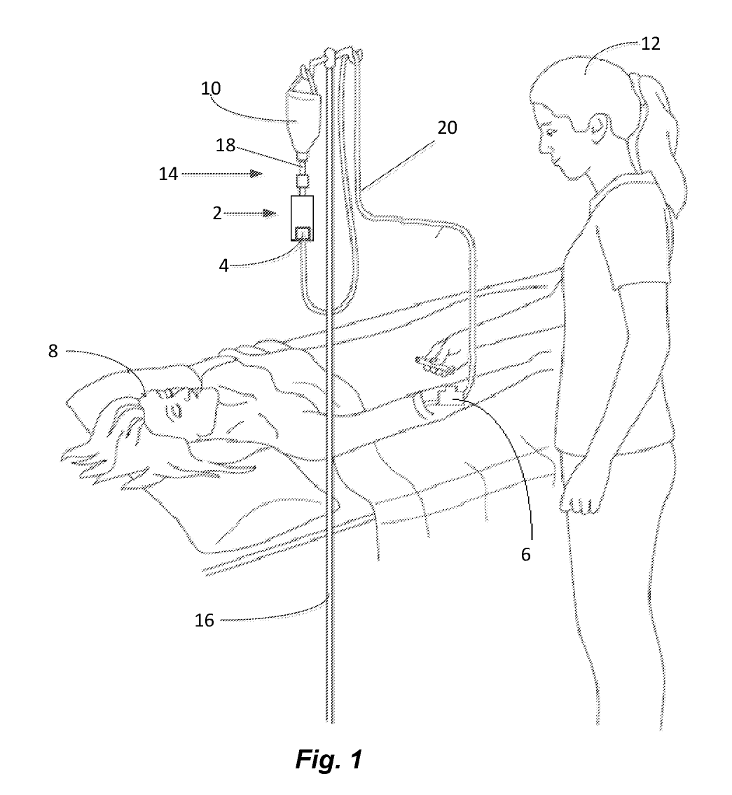

FIG. 1 schematically shows one illustrative use of a drug delivery device 2 configured in accordance with illustrative embodiments of the invention. The drug delivery device 2 may serve as the primary packaging of a drug 4 that needs to be reconstituted prior to intravenous infusion. To that end, in this example, a catheter 6 connects with a patient's vein (the patient is identified by reference number 8). Adhesive tape or similar material may be coupled with the catheter 6 and the patient's 8 arm to ensure that the needle remains in place.

Prior art reconstitution and administration methods generally require multiple steps. Thus, the prior art methods have high potential for error and may introduce pathogens or contaminants from errors in aseptic technique, especially when large doses of drugs 4 are reconstituted. Some embodiments of the invention provide fewer steps and thus reduce the likelihood of contaminating the drug 4 and solution, relative to the prior art. Prior art reconstitution methods are especially unequipped to handle reconstitution of large quantities of drug 4. For example, to reconstitute 20 grams of drug 4 (e.g., lyophilized powder) may require approximately 500 ml of solution, which is the volume of some entire IV bags 10. Transferring this solution using a syringe is time consuming and may create contamination issues. Furthermore, adding the reconstituted medicinal product to an IV bag 10 may pop the bag 10, or may require a draining step. Illustrative embodiments of the device 2 provide advantages over the prior art.

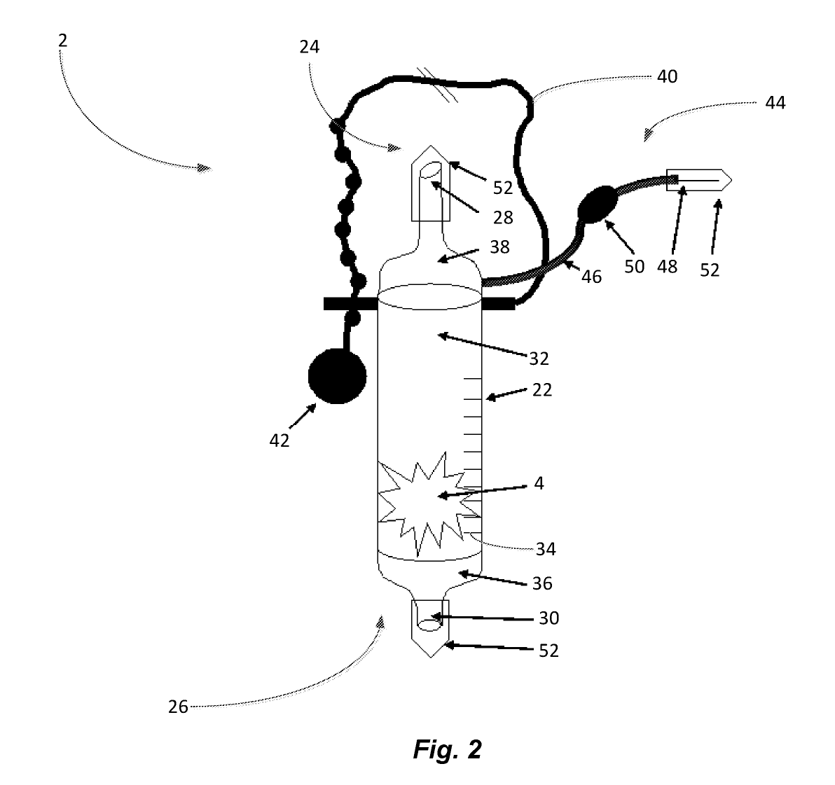

After the device 2 is in place, a nurse, doctor, pharmacist, technician, practitioner, or other user (schematically identified by reference number 12) may intravenously deliver medication to the patient 8, who is lying in a hospital bed. To that end, the nurse 12 may remove a protective cover of the spike (spike cover 51 in FIG. 2) and/or remove a protective cover of the bottom surface (port cover 53 in FIG. 20). Additionally, or alternatively, the nurse 12 may swab the top surface (e.g., a spike—shown in FIG. 2) and/or the bottom surface (e.g., a port—shown in FIG. 2) of the device 2 to remove contaminants prior to connecting the device 2 (e.g., in embodiments where the device 2 is reusable). Next, the nurse 12 may use a medical instrument (e.g., a syringe having a distally located blunt, luer tip complying with ANSI/ISO standards) to provide medication into the device 2, or the device 2 may come pre-packaged with drug 4. For example, in some embodiments where the device 2 is reusable, the medical practitioner 12 may position the drugs 4, such as apotransferrin or transferrin or other intravenous medication, into the device 2.

The device 2 may receive the drug 4 and/or fluids from other means, such as through a gravity feed system 14. In general, traditional gravity feeding systems 14 often have a bag 10 (e.g., the IV fluid bag 10 or bottle) containing a fluid (e.g., saline solution, or anesthesia medication) to be introduced into the patient 8. In some embodiments, the IV fluid bag 10 may be any pharmaceutically acceptable aqueous diluent. Well-known pharmaceutically acceptable aqueous diluents include dextrose solutions (e.g., a 5% dextrose solution), physiological saline solution, (e.g., a 0.9% saline solution), Ringer's solution, lactated Ringer's solution, and solutions containing electrolytes. In some embodiments, the device 2 does not have to be inverted in order for fluid transfer to begin from the device 2 to the patient 8, as required by some prior art devices that are not directly in-line (e.g., prior art that is in-parallel with the line that runs from the fluid bag to the patient). The in-line arrangement allows the drug 4 to be reconstituted and delivered to the patient 8 without the requirement of additional steps or additional tubing. The bag 10 (or bottle) typically hangs from a pole 16 to allow for gravity feeding. The medical practitioner 12 then connects the bag 10 to the device 2 using the spike built into the device 2, or through intermediary tubing 18. The device 2 is fluidly connected to the patient 8 using tubing 20 having an attached spike.

In illustrative embodiments, the spike of the tubing 20 may have a luer taper that complies with the ANSI/ISO standard. After the tubing 20 is connected to the device, gravity and/or a pump causes the fluid to begin flowing into the patient 8. In some embodiments, the feeding system 14 may include shut-off devices on the tubing 20 (e.g., stop-cocks, clamps, and/or locks) to stop fluid flow without having to disconnect the tubing 20 from the device 2.

FIG. 2 schematically shows the drug delivery 2 device of FIG. 1. As mentioned above, the device 2 may contain the drug 4 (also referred to as medication 4). Specifically, the device 2 has a container 22 (e.g., a cylinder 22) that houses the drug 4 in a sterile environment. The container 22 may be formed from polypropylene, polyethylene terephthalate, high density polyethylene, polyvinyl chloride, polystyrene, polylactide, glass, or any other material that is stable and compatible with pharmaceuticals (e.g., hard plastic materials such as polyethylene and polycarbonate). In some embodiments, the device 2 may be tamper-evident and/or tamper-proof. The device 2 has a piercing end 24 and a pierceable end 26. The piercing end 24 is configured to form a fluid connection with (e.g., by piercing) the IV fluid bag 10. To that end, the piercing end 24 may have a spike 28 that is inserted into the port of the IV fluid bag 10. The spike 28 allows the device 2 to be inserted in-line with standard IV fluid bags 10. Furthermore, the pierceable end 26 is configured to form a fluid connection with (e.g., by being pierced by) the IV tubing 20. To that end, the pierceable end 26 may have a port 30 into which a standard IV tubing 20 spike may be inserted. The port 30 allows the device 2 to be inserted in-line with standard IV tubing 20. Although the device 2 is described as having these two ends 24 and 26, in some embodiments, the spike 28 of the piercing end 24 and the port 30 of the pierceable end 26 may be on the same end.

Thus, the device 2 may reconstitute a large quantity of the drug 4 using standard equipment readily available to medical practitioners 12. To that end, in some embodiments, the container 22 may have a corresponding large volume. For example, the container 22 may have a volume of 250 mL, 500 mL, 750 mL, 1000 mL, 1250 mL, 1500 mL, 1750 mL, 2000 mL, 2250 mL or 2500 mL. In some embodiments the container 22 may have a volume of at least 500 mL and/or less than 2500 mL. In other embodiments, the container 22 may have a volume of at least 1000 mL and/or less than 2000 mL. Further embodiments may have a volume of at least 1250 mL and/or less than 1750 mL. The volume is the available space inside the container 22 that can hold, for example, the drug 4 and the fluid.

The various sized containers 22 described above may accommodate a variety of different fluid 32 volumes. While the containers 22 may be able to accommodate a certain volume, it is not necessary that they be entirely filled. For example, a 2000 mL container may be used to contain 500 mL of fluid 32. To that end, in some embodiments, the container 22 may be made of transparent and/or translucent material and have accurate graduations 34 on the surface that indicate volumes including, for example, 250 mL, 300 mL, 400 mL, 500 mL, 600 mL and 700 mL. It should be understood that the markings 34 are just exemplary and that the volume of the container 22 is not limited by the volume markings 34. It should also be understood that although the container 22 is shown with fluid 32, illustrative embodiments do not necessarily come prepackaged with fluid 32. The fluid 32 is shown merely to facilitate discussion of illustrative embodiments and not intended to limit various embodiments of the invention.

The various sized containers 22 are suitable for reconstituting various amounts and kinds of drug 4, for example, apotransferrin or transferrin. In illustrative embodiments, the container 22 houses more than a gram of the drug 4. More specifically, the container 22 may house between 1-5 grams, 5-10 grams, 10-15 grams, or 15-20 grams of the drug 4.

In some embodiments, the amount of the drug 4 prepackaged in the container 22 may correspond to the volume of the container 22. For example, 20 grams of the drug 4 in a 1000 mL container 22, 15 grams of the drug 4 in a 750 mL container 22, 10 grams of the drug 4 in a 500 mL container 22, 5 grams of the drug 4 in a 250 mL container 22, 2.5 grams of the drug 4 in a 125 mL container 22, 1 gram of the drug 4 in a 50 mL container 22. Although the ratio of the weight of the drug 4 in grams to the volume of the container 22 is 1:50 in the above described embodiments, illustrative embodiments of the invention are not limited to this ratio. The ratio of the weight of the drug 4 in grams to the volume of the container 22 in mL may be, for example, greater than 1:40 and/or between about 1:40 and about 1:60.

In some embodiments, the container 22 may be a cylinder 22. The cylinder 22 may have a tapered bottom 36 and a tapered top 38.

The device 2 may have a fluid control mechanism 44. The fluid control mechanism 44 controls the flow and/or rate of flow of fluid 32 into and the container 22. In illustrative embodiments the fluid control mechanism 44 includes a flexible tube 46. The flexible tube 46 may end in a fitting to accept a standard hypodermic needle 48 (e.g., a luer-lock fitting). The needle 48 may be, for example, threaded onto the end of the flexible tube 46. After the needle 48 has been coupled with the flexible tube 46, it may be inserted into the needle port on the IV fluid bag 10 (needle port not shown).

In some embodiments, the device 2 may include a lock (e.g., a tubing slide clamp) and/or a pump 50 (e.g., a bulb pump) to modulate fluid flow. Although the lock and pump are both shown as reference numeral 50, it should be understood that they may come as separate components rather than as a single component. The lock and/or pump 50 may come as part of the flexible tubing 46. After the needle 48 has been inserted into the needle port, the lock 50 on the flexible tubing 46 may be opened, and the pump 50 (e.g., bulb pump) can be used to start the flow of fluid 32 into the container 22 from the fluid bag 10.

In some embodiments, the device 2 may have a strap 40 that is securely attached to both sides of the container 22. The tightness of the strap 40 may be adjustable to facilitate the compatibility of the device below a range of standard IV fluid bags 10. The strap 40 may help support the weight of the device 22 on the IV pole 16. Furthermore, the strap 40 is configured to support the weight of the container 22 when the container is full of fluid 32. The strap 40 may have a safety mechanism 42 to prevent accidental detachment of the device 2 from the IV pole 16.

Several non-limiting examples of drug 4 doses and dosing schedules are provided below. As described above, the drug 4 may be apotransferrin such as recombinant apotransferrin (e.g., Optiferrin®).

In some embodiments, a single dose may comprise 5 grams of drug 4 reconstituted in 250 ml, 500 ml, 750 ml, or 1000 ml of fluid 32, or in a range between any of the above described volumes (e.g., 250 mL-1000 mL, 500 mL-750 mL; 250 mL-750 mL, etc.). The single dose may be administered, for example, over the course of one day once every four weeks, once every three weeks, once every two weeks, once per week, twice per week, three times per week, or four times per week or more.

In other embodiments, a single dose may comprise 10 grams of drug 4 reconstituted in 250 ml, 500 ml, 750 ml, or 1000 ml of fluid 32 or in a range between any of the above described volumes. The single dose may be administered, for example, over the course of one day once every four weeks, once every three weeks, once every two weeks, once per week, twice per week, three times per week, or four times per week or more.

In other embodiments, a single dose may comprise 15 grams of drug 4 reconstituted in 500 ml, 750 ml, or 1000 ml of fluid 32 or in a range between any of the above described volumes. The single dose may be administered, for example, over the course of one day once every four weeks, once every three weeks, once every two weeks, once per week, twice per week, three times per week, or four times per week or more.

In other embodiments, a single dose may comprise 20 grams of drug 4 reconstituted in 500 ml, 750 ml, or 1000 ml of fluid 32, or in a range between any of the above described volumes. The single dose may be administered, for example, over the course of one day once every four weeks, once every three weeks, once every two weeks, once per week, twice per week, three times per week, or four times per week or more.

In some embodiments, a single dose may comprise 5 grams of drug 4 reconstituted in 250 ml, 500 ml, 750 ml, 1000 ml, 1250 ml, 1500 ml, 1750 ml, or 2000 ml of fluid 32, or in a range between any of the above described volumes (e.g., 500 mL-2000 mL, 750 mL-1500 mL; 1000 mL-2000 mL, etc.). The single dose may be administered, for example, over the course of two days once every four weeks, once every three weeks, once every two weeks, once per week, twice per week, or three times per week or more.

In other embodiments, a single dose may comprise 10 grams of drug 4 reconstituted in 250 ml, 500 ml, 750 ml, 1000 ml, 1250 ml, 1500 ml, 1750 ml, or 2000 ml of fluid 32, or in a range between any of the above described volumes. The single dose may be administered, for example, over the course of two days once every four weeks, once every three weeks, once every two weeks, once per week, twice per week, or three times per week or more.

In other embodiments, a single dose may comprise 15 grams of drug 4 reconstituted in 500 ml, 750 ml, 1000 ml, 1250 ml, 1500 ml, 1750 ml, or 2000 ml of fluid 32, or in a range between any of the above described volumes. The single dose may be administered, for example, over the course of two days once every four weeks, once every three weeks, once every two weeks, once per week, twice per week, or three times per week or more.

In other embodiments, a single dose may comprise 20 grams of drug 4 reconstituted in 500 ml, 750 ml, 1000 ml, 1250 ml, 1500 ml, 1750 ml, or 2000 ml of fluid 32, or in a range between any of the above described volumes. The single dose may be administered, for example, over the course of two days once every four weeks, once every three weeks, once every two weeks, once per week, twice per week, or three times per week or more.

The drug 4 dose and dosing schedule required by the patient 8 may be adjusted as necessary. For example, in a patient suffering from a transferrinemia, the drug 4 may be apotransferrin and the dose and dosing schedule may be adjusted based on the patient's 8 serum transferrin levels. In illustrative embodiments, the patient's 8 dose and dosing schedule may be adjusted to achieve a serum transferrin level of 80-500 mg/dL. The patient's 8 dose and dosing schedule may be adjusted to achieve a serum transferrin level of 100-450 mg/dL. Furthermore, the patient's 8 dose and dosing schedule may be adjusted to achieve a serum transferrin level of 150-400 mg/dL. The patient's 8 dose and dosing schedule may also be adjusted to achieve a serum transferrin level of 170-370 mg/dL.

The drug 4 dose and dosing schedule may be adjusted as necessary. For example, in a patient suffering from a transferrinemia, the drug 4 may be apotransferrin and the dose and dosing schedule may be adjusted based on the patient's 8 serum hemoglobin levels. In illustrative embodiments, the patient's 8 dose and dosing schedule may be adjusted to achieve a serum transferrin level of 8-22 g/dL. The patient's 8 dose and dosing schedule may be adjusted to achieve a serum transferrin level of 10-20 g/dL. Furthermore, the patient's 8 dose and dosing schedule may be adjusted to achieve a serum transferrin level of 12-18 mg/dL. The patient's 8 dose and dosing schedule may also be adjusted to achieve a serum transferrin level of 13-15 g/dL.

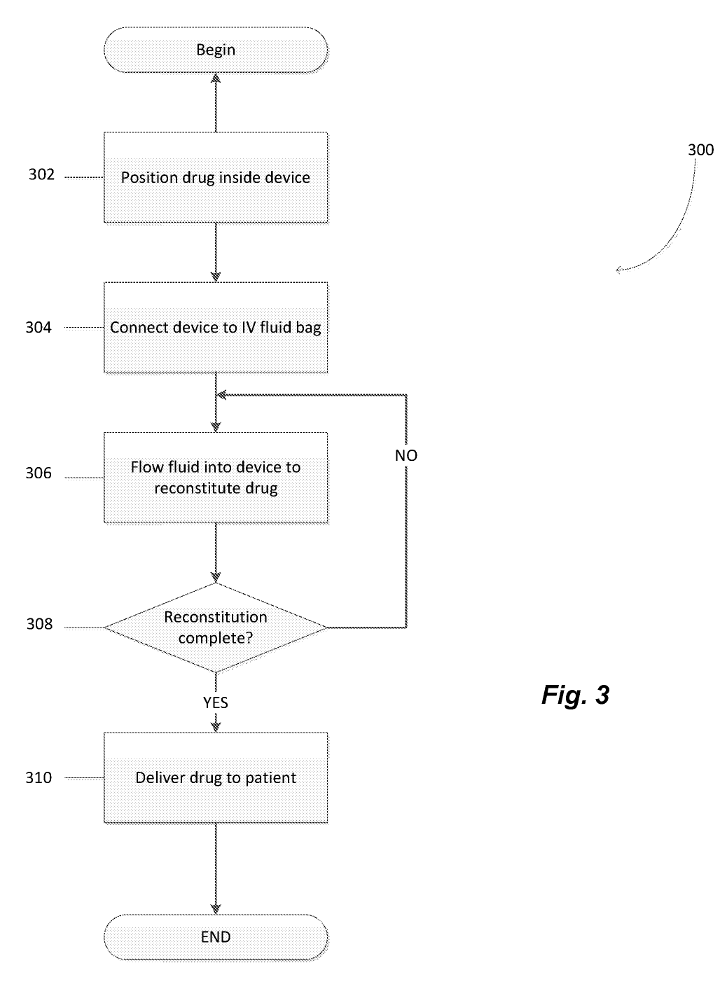

FIG. 3 shows a process 300 of delivering a drug 4 to a patient 12 in accordance with illustrative embodiments of the invention. The process 300 begins at step 302, in which the drug 4 is positioned inside the device 2. Specifically, the drug 4 is positioned within the container 22 of the device 2. As described above, the drug 4 may come prepackaged within the container 22, or a medical practitioner 12 can place the drug 4 inside the container 22. In some embodiments, the drug 4 is lyophilized. In some embodiments, the drug 4 is a concentrated liquid or syrup. A variety of drugs 4 may be placed inside the container 22. Preferably, the drug 4 is a lyophilized-recombinant protein replacement for apotransferrin. In some embodiments, lyophilized apotransferrin may be in combination with another drug 4. Furthermore, because of the large capacity of the container 22, a large amount of the drug 4 can be placed inside the container 22 for reconstitution.

After the drug 4 has been positioned inside the device 2, the process proceeds to step 304, which connects the device 2 to the IV fluid bag 10. The medical practitioner 12 may begin by removing the outer packaging of the device 2 if any was provided. The practitioner 12 may then hang the device 2 from the IV pole 16 below the IV fluid bag 10 containing the fluid solution 32. The container 22 may include a strap 40 that is securely attached to the IV pole 16. The strap 40 allows the device 2 to hang below a range of standard IV fluid bag 10 sizes. The strap 40 may be adjustable and configured to support the weight of the device 2 when it is full of medical fluid 32. Furthermore, in some embodiments, the strap 40 may have a safety mechanism 42 to prevent accidental detachment.

The spike 28 of the device 2 may pierce the port of the IV fluid bag 10 (e.g., by pressing the spike 28 into the port). In some embodiments a protective cap 51 has to be removed from the spike 28 before the spike can pierce the port. Furthermore, the medical practitioner 12 may swab the port of the IV fluid bag 10, and/or the spike 28 of the device 2 to reduce the risk of introducing a microbial infection into the line.

The process then moves to step 306, which flows fluid 32 into the device 2 to reconstitute the drug 4. Fluid from the IV bag 10 may not initially flow into the device 2, or it may flow at an undesired rate. The practitioner 12 can control the rate of fluid flow to be faster, slow, or stopped using the fluid control mechanism 44. This may be accomplished by, for example, by using a hypodermic needle 48 to pierce the needle port of the IV fluid bag 10. In illustrative embodiments, the device 2 may include the flexible tube 46 that has the pump 50 and ends in a hypodermic needle 48. In some embodiments, the hypodermic needle 48 may also have a protective cap 55. After the hypodermic needle 48 is inserted into the needle port of the IV fluid bag 10, the practitioner 12 may use the pump 50 to begin the flow of fluid 32 from the IV fluid bag 10 into the container 22. In illustrative embodiments having the slide lock 50 that prevents activation of the pump 50, the lock 50 is opened prior to pumping the fluid 32 into the container 22. The slide lock 50 can also be used to stop the flow of fluid 32 into the container 22.

The practitioner 12 may use the pump 50 to control the flow of fluid 32 into the device 2 until the fluid reaches a predetermined gradient 34. The practitioner 12 may also use the entire contents of the IV fluid bag 10.

The process then determines if the reconstitution is complete (step 308). For example, the practitioner 12 can visually inspect the container 22 to determine whether the lyophilized drug 4 has been reconstituted. The practitioner 12 may gently swirl and/or gently invert the device 2 to aid in the reconstitution process. If the reconstitution is not complete, the process returns to step 306 and provides more fluid 32 into the container 22 of the device 2 to further reconstitute the drug 4 (e.g., by using the fluid control mechanism 44). In some embodiments, the entire contents of the IV fluid bag 10 may be dispensed into the container 22. Some embodiments may require additional fluid beyond what is provided in a single fluid bag 10. The container 22 may be gently swirled and/or inverted to mix and reconstitute the drug 4. In some embodiments, the device 2 may include a vortex mixer (e.g., motorized) to help reconstitute the drug 4. The medical practitioner 12 may visually inspect the solution to determine whether the drug 4 is sufficiently reconstituted. To that end, the container 22 may be a least partially transparent. In some embodiments, the process waits until the drug 4 is fully in solution before moving to the next step.

If the reconstitution is complete, the process proceeds to step 310, which delivers the drug 4 to the patient 8. Thus, illustrative embodiments of the invention advantageously allow the reconstitution of a drug 4 in-line with a standard IV fluid bag 10 (i.e., no special or modified IV fluid bag 10 is required). Additionally, the device 2 allows for the reconstitution and delivery of a high-dosage of lyophilized drug 4. The process proceeds to step 310, which delivers the drug 4 to the patient 8.

To deliver the drug 4 to the patient 8, the device 22 is connected to the IV fluid line 20. The port 30 of the device may pierced by the spike of the IV fluid line 20. In some embodiments, the protective cap 53 has to be removed from the port 10 prior to forming this fluid connection. Furthermore, the medical practitioner 12 may swab the port 30 of the device 2 and/or the spike of the IV fluid line 20 to reduce the risk of introducing a microbial infection into the line.

The rate and amount of fluid delivery to the patient 8 can be controlled via standard IV practices known in the art using standard IV tubing 20. The tubing 20 may have, for example, slide locks and drip chambers and other fluid control mechanisms that are more commonly found on IV tubing 20. Thus, the entire contents of the container 22 may be administered to the patient 12, or the solution can be drained through the IV line 20 until the desired amount of the solution remains in the container 22 to provide a partial dose. In some embodiments, when the cylinder 22 is empty, the attached IV bag 10 is fully collapsed and provides negative pressure to prevent air from being infused to the patient 8. In some embodiments, the patient 12 may be subjected to a dosing schedule. Sufficient quantities of the drug 4 can be newly reconstituted based on the dosing schedule, or a large amount of drug 4 can be reconstituted to cover multiple dosings, as described above with regard to dosing schedules.

It should be noted that this process is a simplified version of a more complex process of delivery of a drug 4 to a patient 8. As such, the actual process may have additional steps that are not discussed. In addition, some steps may be performed in a different order, or in parallel with each other. Accordingly, discussion of this process is illustrative and not intended to limit various embodiments of the invention. Moreover, although this process is discussed with regard to delivering a single drug 4, the process of FIG. 3 can also be used to deliver more than one drug 4. Indeed, rather than just deliver a single drug 4, those in the art can modify the process to deliver a plurality of drugs 4 simultaneously, e.g., by providing multiple drugs 4.

Although the above discussion discloses various exemplary embodiments of the invention, it should be apparent that those skilled in the art can make various modifications that will achieve some of the advantages of the invention without departing from the true scope of the invention.

### Example 1: Method of Treating an Atransferrinemia Patient in Accordance with Illustrative Embodiments of the Device

The following prophetic example is illustrative of treating a patient 8 suffering from atransferrinemia by delivering apotransferrin 4 to the patient 8 using the device 2 of the invention.

An adult patient 8 was diagnosed as suffering from atransferrinemia after presenting with fatigue. A blood test revealed that the patient 8 suffered from microcytic anemia and the patient's 8 serum transferrin levels were 10 mg/dL, well below the normal range of 170-370 mg/dL for a healthy adult. Serum transferrin levels may be determined by any method known in the art, e.g., by an immunoassay such as the ELISA assay (Vanarsa Arthritis Res Ther. 2012 Aug. 7; 14(4):R182).

The patient 8 starts a treatment of apotransferrin using an illustrative embodiment of the device 2. An initial dose of 5 grams of apotransferrin 4 reconstituted in 250 ml of 0.9% saline 32 is administered once per week.

A prepackaged 500 ml cylinder 22 containing 5 grams of lyophilized recombinant apotransferrin 4 is connected to an IV fluid bag 10 and reconstituted in 250 ml of 0.9% saline 32 in accordance with illustrative embodiments of the present invention. The reconstituted apotransferrin 4 is delivered intravenously to the patient 8 over the course of 2 hours.

Five days later, the patient's 8 blood serum levels of transferrin are tested and determined to be 50 mg/dL.

One week after the patient's 8 first dose of apotransferrin 4, a second dose of 5 grams of apotransferrin 4 is provided to the patient 8 in the same manner. Five days later, the patient's 8 blood serum transferrin levels are again found to be below the normal range for a healthy adult.

5 grams of apotransferrin 4 are then administered to the patient in the same manner, once weekly. After eight weeks of treatment, the patient's 8 blood serum levels of transferrin are determined to be at a steady state level of 70 mg/dL, still well-below the normal range for a healthy adult.

The patient's 8 dose is adjusted to a higher dose of apotransferrin 4 using the device 2 of the invention. Next, a dose of 10 grams of apotransferrin 4 reconstituted in 500 ml of 0.9% saline 32 is administered once per week.

A prepackaged 500 ml cylinder 22 containing 10 grams of lyophilized recombinant apotransferrin 4 is connected to an IV fluid bag 10 and reconstituted in 500 ml of 0.9% saline 32 in accordance with illustrative embodiments of the present invention. The reconstituted apotransferrin 4 is delivered intravenously to the patient 8 over the course of 4 hours.

Five days later, the patient's 8 blood serum levels of transferrin are tested and determined to be 130 mg/dL.

The patient 8 continues to be treated once per week with 10 grams of apotransferrin 4, reconstituted in accordance with illustrative embodiments of the present invention, as described above. After eight weeks of treatment, the patient's 8 serum transferrin levels are found to be at a steady state level of 178 mg/dL, which is within the normal range for a healthy adult.

The patient 8 continued to be treated once per week with 10 grams of apotransferrin 4, reconstituted in accordance with illustrative embodiments of the present invention. Blood serum levels of transferrin are routinely monitored and found to remain at steady state levels within the normal range for a healthy adult.

No further dose adjustments are made for the patient 8, and the patient 8 continues to be treated with 10 grams of apotransferrin 4, once weekly, as described above, for life.

This example is solely for illustrative purposes, and not intended to limit all illustrative embodiments of the invention. A person of skill in the art would understand that illustrative embodiments may operate on variations of the disease, and that various drugs, diluents, doses, and dosing schedules may be used.

What is claimed is:

1. A device that houses a lyophilized drug to be reconstituted and administered to a patient having a disease, the device comprising:
a transparent cylinder configured to house the lyophilized drug, the cylinder having a volume of at least 250 mL,
the cylinder having a first end opposite a second end, the first end having a first spike configured to pierce an IV fluid bag to form a fluid connection between the cylinder and the IV fluid bag,
the second end having a port configured to be pierced by a second spike that is part of a tubing to form a fluid connection between the cylinder and the tubing;
at least a portion of the volume of the cylinder being occupied by at least 5 grams of the lyophilized drug.

2. The device as defined by claim 1, wherein the drug is apotransferrin and the disease is selected from the group consisting of a transferrinemia, hepatic failure, thalassemia, iron overload, hemochromatosis, and age-related macular degeneration.

3. The device as defined by claim 1, wherein the device is provided in a sterile packaging with a premeasured amount of the drug in the cylinder.

4. The device as defined by claim 1, wherein the cylinder is fluidly connected with the IV fluid bag so as to facilitate reconstitution of the lyophilized drug.

5. The device as defined by claim 4, wherein the tubing is connected to a patient and the cylinder is fluidly connected with the tubing so as to facilitate delivery of the drug to the patient.

6. The device as defined by claim 1, wherein the device is provided in tamper-evident packaging.

7. The device as defined by claim 1, wherein the volume of the cylinder is between about 250 mL and about 1000 mL.

8. The device as defined by claim 2, wherein apotransferrin is at a weight between about 5 grams and 20 grams.

9. A method of reconstituting a lyophilized drug that is to be administered to a patient having a disease, the method comprising:
providing a device comprising a transparent cylinder having at least 5 grams of lyophilized drug, the cylinder having a volume of at least 250 mL,
the cylinder having a first end opposite a second end, the first end having a first spike configured to pierce an IV fluid bag to form a fluid connection between the cylinder and the IV fluid bag,
the second end having a port configured to be pierced by a second spike that is part of a tubing to form a fluid connection between the cylinder and the tubing;
piercing the IV fluid bag so as to form a fluid connection between the cylinder and the IV fluid bag; and
reconstituting the drug in the cylinder to form a reconstituted drug.

10. The method as defined by claim 9, wherein the drug is apotransferrin and the disease is atransferrinemia.

11. The method as defined by claim 9, further comprising:
providing the device in a sterile packaging with a premeasured amount of the drug in the cylinder.

12. The method as defined by claim 9, wherein the volume of the cylinder is between about 250 mL and about 1000 mL.

13. The method as defined by claim 9, wherein the drug in the cylinder is apotransferrin and weighs between about 5 grams and 20 grams.

14. A method of treating a patient having a disease, comprising administering to the patient in need of such treatment an effective amount of an apotransferrin preparation using the device of claim 1, wherein the preparation comprises about 1-20 grams of apotransferrin.

15. The method of claim 14, wherein the disease is selected from the group consisting of atransferrinemia, hepatic failure, thalassemia, iron overload, hemochromatosis, and age-related macular degeneration.

16. The method of claim 14, wherein the apotransferrin preparation comprises a recombinant protein replacement for apotransferrin.

17. The method of claim 14, wherein the treatment produces a serum transferrin level in the patient of about 150-400 mg/dL.

18. The method of claim 14, wherein the treatment produces a serum hemoglobin level in the patient of at least 10 g/dL.

19. The method of claim 14, wherein the treatment is administered to the patient once every week.

20. The method of claim 14, wherein the treatment is administered to the patient once every two weeks.
